(19) United States
(12) Patent Application Publication
(10) Pub. No.: US 2009/0143458 A1
(43) Pub. Date: Jun. 4, 2009

(54) USE OF ACE INHIBITORS AND/OR ANGIOSTENSIN II RECEPTOR ANTAGONISTS FOR THE IMPROVING AND/OR MAINTAINING THE SKIN TONE AND FOR THE TREATMENT OF SKIN AGEING

(75) Inventors: Benny V. Jensen, Roskilde (DK); Richard Bonnichsen, Mahe (SC)

Correspondence Address: GIFFORD, KRASS, SPRINKLE,ANDERSON & CITKOWSKI, P.C, PO BOX 7021, TROY, MI, 48007-7021, US

(73) Assignee: Ace ApS, Helsingør (DK)

(21) Appl. No.: 10/597,545

(22) PCT Filed: Jan. 28, 2005
(86) PCT No.: PCT/DK2005/000065

§ 371 Date: Oct. 11, 2007

Related U.S. Application Data
(60) Provisional application No. 60/553,661, filed on Mar. 16, 2004.

(30) Foreign Application Priority Data
Jan. 30, 2004 (DK) PA 2004 00136

(51) Int. Cl. A61K 31/40

(52) U.S. Cl. 514/423

(57) ABSTRACT

The present invention relates to use of an ACE inhibitor and/or angiotensin II receptor antagonist of the preparation of a medicament for the treatment of skin ageing or wrinkling. Furthermore, the present invention relates to use of an ACE inhibitor and/or angiotensin II receptor antagonist for the preparation of a cosmetic composition.

This application is a nonprovisional of U.S. provisional application Ser. No. 60/553,661 filed 15 Mar. 2004, which is hereby incorporated by reference in its entirety. The application claims priority from Danish patent application number PA 2004 00136 filed 30 Jan. 2004, which is hereby incorporated by reference in its entirety. All patent and nonpatent references cited in the application, or in the present application, are also hereby incorporated by reference in their entirety.

### FIELD OF INVENTION

The present invention relates to cosmetic compositions comprising at least one ACE inhibitor and/or angiotensin II receptor antagonist, or a cosmeceutically acceptable salt thereof. The present invention also provides cosmetic methods for improving and/or maintaining the skin tone of an individual, said method comprising contacting the skin of said individual with the composition described herein. The present invention also relates to use of an ACE inhibitor and/or angiotensin II receptor antagonist for the preparation of a medicament for the treatment of skin ageing or wrinkling. Furthermore, the present invention relates to use of an ACE inhibitor and/or angiotensin II receptor antagonist for the preparation of a cosmetic composition.

### BACKGROUND OF INVENTION

### Skin and Ageing

The skin dermis makes up 90% of the thickness of the skin and consists of a three-dimensional extracellular matrix (ECM) of loose connective tissue composed of highly stable fibers of collagen and elastin. Collagen is the major constituent of skin and constitutes more than 70% of the mass of the skin in terms of its dry weight. Collagens are synthesized by fibroblasts, and comprise a large family of glycoproteins which are located in the extracellular matrix. 20 different types of collagens (types I-XX) have been defined so far, and fibrillar collagens type I and II predominate in the skin (Epstein and Munderloh, J. Biol. Chem. 253:1336, 1978; Fukar et al., Acta Derm. Venereol. 68:196, 1988; Clore et al., Biochim. Biophys. Acta 586:384, 1979; Chan and Cole, Anal. Biochem. 139:322, 1984). In young skin, collagen molecules stay soluble and slide over one another, giving skin its softness, strength, resiliency and preventing skin tearing—a problem in aged individuals with reduced collagen content.

Skin ageing processes act synergistically to alter the structure, organization, and composition of elastin and collagen. These changes manifest themselves externally by signs of ageing, such as lines, wrinkles, loss of elasticity, sagging, skin dryness and unevenness, blotches, age spots, pigmented spots, and benign and malignant neoplasms. A substantial amount of evidence indicates that these skin changes are caused by changes to the synthesis and degradation of skin collagen. As skin ages, fibroblasts lose their ability to react to growth factors for the proliferation and synthesis of collagen, and the dermis and the epidermis become thin (West, Arch. Dermatol. 130:87, 1994). Examination by scanning electron microscopy reveals a decrease in the number of collagen fibre bundles in normal human skin with age (Lovell et al., Br. J. Dermatol. 117:419, 1987). It has been found that collagen synthesis decreases in a statistically significant linear manner with age (Dumas et al., C. R. Acad. Sci. 319:1127, 1996; Phillips et al., J. Invest. Dermatol. 103, 228, 1994; Uitto and Bernstein, J. Investig Dermatol. Symp. Proc. 3:41, 1998).

Ageing of skin is accelerated during the menopause, when there are alterations in the derma elastic tissue, a decrease in the rate of collagen formation rate and in the derma thickness. This leads, in the menopausal woman, to the skin and/or the mucous membranes becoming thinner. The woman then has the feeling of a “dry skin” or drawn skin with reduced suppleness and an increase of fine wrinkles and small surface wrinkles can also be noticed.

### Premature Ageing

Although ageing is thought to be a genetically programmed phenomenom, environmental factors, such as ultraviolet (UV) radiation from sunlight, chemicals, cigarette smoking, high sugar diets, diseases (which also may have a genetic cause), medical treatments and air pollution, may also cause premature skin ageing. Damage to skin collagen and elastin is the hallmark of long-term exposure to UV radiation (“photo-ageing”). Collagen synthesis is reduced in photoaged skin by approximately 45% compared to protected skin (Kligman et al., Photodermatol. Photoimmunol. PhoPhotomed. 16:224, 2000). UV irradiation accelerates the disappearance of collagen contents in the photoaged skin (Fisher et al, J. Invest. Dermatol. 117:219, 2001).

Photoaging can be characterized histologically by diminution of collagen, ultrastructural alterations of collagen fibrils, and accumulation of elastotic material in the papillary dermis. Clinically, photoageing is characterized by coarseness, wrinkling, mottled pigmentation, laxity, telangiectasia, lentigenes, and benign as well as malignant neoplasms. UV irradiation acts in an additive manner with tobacco smoke to further speed premature aging of human skin.

Decreased collagen fibre renewal and/or other signs of skin ageing are also associated with medical syndromes, for example syndrome X, diabetes, Hutchinson-Gilford progeria, Werners syndrome, Kindler syndrome, corticoid hormone hyper-secretion or vitamin deficiencies, in particular vitamin C deficiency. Medical treatments may also decrease collagen fibre renewal, for example radiation therapy, glucocorticoid administration or administration or vitamin D or derivatives thereof.

### The Renin-Angiotensin-Aldosterone System

The renin-angiotensin-aldosterone system plays an integral role in the pathophysiology of hypertension by affecting the regulation of fluid volume, electrolyte balance and blood volume. Renin catalyzes the conversion of angiotensinogen into an inactive substance, angiotensin I. Angiotensin-converting enzyme (ACE) then converts angiotensin I to the physiologically active angiotensin II, which binds the AT1 and/or AT2 angiotensin receptor and causes potent vasoconstriction, aldosterone secretion and sympathetic activation (Berstein K E, Berk B C, “The biology of angiotensin II receptors” Am J Kid Dis 1993).

ACE inhibitors are used for treatment of hypertension, heart failure and nephropathy. Evidence suggests that angiotensin receptor antagonists may be just as effective as an angiotensin converting enzyme (ACE) inhibitor in treating patients who are at high risk of cardiovascular events after myocardial infarction (BMJ. 2003 Nov. 15; 327(7424):1123). ACE inhibitors may also slow the progress of diabetic kidney disease in middle-aged persons with type 2 diabetes (Annals of Internal Medicine 1999; 131:660-667, 707-708.)

### Effect of the Renin-Angiotensin System on Collagen

Angiotensin II stimulates collagen type I formation by activation of the collagen I gene (Tharaux P L, et al., “Angiotensin II activates collagen I gene through a mechanism involving the MAP/ER kinase pathway”, Hypertension 2000, 36(3):330-336). Collagen type I gene expression increases after vascular injury, however using ACE inhibitors or angiotensin II antagonists decreases levels of gene expression significantly. (Patten R D et al., “Effects of angiotensin II receptor blockade versus angiotensin-converting-enzyme inhibition on ventricular remodelling following myocardial infarction in the mouse”, Clin Sci (Lond). 2003 February; 104(2):109-18). Activation of the renin-angiotensin-aldosterone system in the myocardial collagen network can lead to progressive collagen accumulation (Brilla C G et al., “Renin-angiotensin system and myocardial collagen matrix remodelling in hypertensive heart disease: in vivo and in vitro studies on collagen matrix regulation”, Clin Investig 1993; 71(5 Suppl):S35-41)

### Current Anti-Ageing Treatments

A large number of different compositions and methods are currently used to ameliorate skin ageing. Both stimulation of procollagen production and reduction of collagen breakdown are seen as key to success in maintaining the integrity of skin (see e.g. US patent application 20010053347). Various cosmetic and/or dermatological compositions containing active agents are available for this, and collagen may also be subcutaneously injected. More invasive treatments include surgical “face-lifts”, botox injections and chemical skin peels.

### SUMMARY OF INVENTION

The skin provides protective functions of importance to our survival. These functions can be detrimentally affected by the changes in the skin structure due to ageing. It is thus desirable to provide medicaments for treatment of skin ageing and/or wrinkling. Furthermore, it is desirable for cosmetic reasons to have youthful-looking skin

The present invention provides cosmetic compositions comprising at least one ACE inhibitor and/or angiotensin II receptor antagonist, or a cosmeceutically acceptable salt thereof, as well as cosmetic methods for improving and/or maintaining the skin tone of an individual. The present invention also relates to use of an ACE inhibitor and/or angiotensin II receptor antagonist for the preparation of a medicament for the treatment of skin ageing and/or wrinkling, and to use of an ACE inhibitor and/or angiotensin II receptor antagonist for the preparation of a cosmetic composition.

It is surprising and unexpected that said inhibitors and antagonists are effective in the compositions and methods disclosed herein, as ACE and angiotensin II are known to be involved in triggering collagen synthesis, whereas teachings within the prior art suggest that ageing skin is associated with a reduction in collagen.

Ageing and prematurely aged skin are not characterised merely by a reduced collagen content in the skin, but also contain foci consisting of collagen deposits. Without being bound by theory, it is hypothesised that the use of an ACE inhibitor or angiotensin inhibitor acts to reduce the uneven collagen deposits in ageing and prematurely aged skin, thus evening out the skin texture. Surprisingly, the compounds of the present invention do not lead to damaging levels of skin collagen reduction, but instead improve and/or maintain the skin tone of an individual.

### DETAILED DESCRIPTION OF THE INVENTION

### ACE Inhibitors and Angiotensin II Receptor Antagonists Suitable for Use in any of the Methods, Uses and Compositions of the Present Invention

Any suitable ACE inhibitor and/or angiotensin II receptor antagonist—or respective pharmaceutically/cosmetically acceptable salt thereof—may be used in any of the methods, uses and compositions of the present invention.

In one embodiment, said ACE inhibitor and/or angiotensin II receptor antagonist may be selected from the following:

Alacepril, Delapril, Benazepril, Cilazapril, Captopril, Enlapril, Fosinopril, Lisinopril, Moexipril, Perindopril, Ramipril, Quinapril, Trandolapril, Imidapril, Isradipin, perindopril, spirapril, temocapril, Enalapril, losartan (Cozaar), valsartan (Diovan), irbesartan (Avapro), candesartan (Atacand), telmisartan (Micardis), eprosartan, tasosartan, zolarsartan, Zofenapril, Isradipin and Candesartancilexetil, or a cosmeceutically-acceptable salt thereof, alatriopril, altiopril calcium, ancovenin, benazepril, hydro-chloride, benazeprilat, benzazepril, benzoylcaptopril, captopril-cysteine, captopriglu-tathione, ceranapril, ceranopril, ceronapril, cilazaprilat, converstatin, delapril-diacid, enalaprilat, enalkiren, enapril, epicaptopril, foroxymithine, fosfenopril, fosenopril, fosenopril sodium, fosinopril sodium, fosinoprilat, fosinoprilic acid, glycopril, hemorphin-4, idapril, indolapril, indolaprilat, libenzapril, lyciumin A, lyciumin B, mixanpril, moexiprilat, moveltipril, muracein A, muracein B, muracein C, perindoprilat, pivalopril, pivopril, quinapril hydrochloride, Pentopril, pentoprilat, quinaprilat, ramiprilat, spirapril, spirapril hydrochloride, spiraprilat, spiropril hydrochloride, temocapril hydrochloride, teprotide, trandolaprilat, utibapril, zabicipril, zabiciprilat, losartan (Cozaar), valsartan (Diovan), irbesartan (Avapro), candesartan (Atacand), telmisartan(Micardis), eprosartan, tasosartan, zolarsartan, Isradipin, Candesartancilexetil, olmesartan, medoxomil, zofenoprilat, Asp-Arg-Val-Tyr-Val-His-Pro-Phe; Asn-Arg-Val-Tyr-Val-His-Pro-Phe; Ala-Pro-Gly-Asp-Arg-Ile-Tyr-Val-His-Pro-Phe Glu-Arg-Val-Tyr-Ile-His-Pro-Phe; Asp-Lys-Val-Tyr-Ile-His-Pro-Phe; Asp-Arg-Ala-Tyr-Ile-His-Pro-Phe; Asp-Arg-Val-Thr-Ile-His-Pro-Phe; Asp-Arg-Val-Tyr-Leu-His-Pro-Phe; Asp-Arg-Val-Tyr-Ile-Arg-Pro-Phe; Asp-Arg-Val-Tyr-Ile-His-Ala-Phe; Asp-Arg-Val-Tyr-Ile-His-Pro-Tyr; Pro-Arg-Val-Tyr-Ile-His-Pro-Phe; Asp-Arg-Pro-Tyr-Ile-His-Pro-Phe; Asp-Ar-Val-Tyr; 2-Ile-His-Pro-Phe; Asp-Arg-norLeu-Tyr-Ile-His-Pro-Phe; Asp-Arg-Val-Tyr-norLeu-His-Pro-Phe; Asp-Arg-Val-homoSer-Tyr-Ile-His-Pro-Phe; Val-Trp.

Thus, said ACE inhibitor and/or angiotensin II receptor antagonist may be selected from Alacepril, Delapril, Cilazapril, Benazepril, Captopril, Enlapril, Fosinopril, Lisinopril, Moexipril, Perindopril, Ramipril, Quinapril, Trandolapril, Imidapril, Isradipin, perindopril, spirapril, temocapril, Pentopril, pentoprilat, Enalapril, Zofenapril, or a pharmaceutically/cosmeceutically acceptable salt thereof. Equally preferably, said ACE inhibitor or angiotensin II receptor antagonist is selected from losartan (Cozaar), Val-Trp, valsartan (Diovan), irbesartan (Avapro), candesartan (Atacand), (Micardis), eprosartan, tasosartan, zolarsartan, Isradipin, Candesartancilexetil losartan (Cozaar), valsartan (Diovan), irbesartan (Avapro), candesartan (Atacand), telmisartan(Micardis), eprosartan, tasosartan, zolarsartan, Val-Trp, Isradipin, Candesartancilexetil and olmesartan medoxomil. Equally, further ACE inhibitors suitable for use in the present invention are any of those disclosed in Patent Application with publication number WO 00/56345 (incorporated herein by reference), such as any of the following:

### Asp-Arg-Val-Tyr-Val-His-Pro-Phe

### Asn-Arg-Val-Tyr-Val-His-Pro-Phe

### Ala-Pro-Gly-Asp-Arg-Ile-Tyr-Val-His-Pro-Phe

### Glu-Arg-Val-Tyr-Ile-His-Pro-Phe

### Asp-Lys-Val-Tyr-Ile-His-Pro-Phe

### Asp-Arg-Ala-Tyr-Ile-His-Pro-Phe

### Asp-Arg-Val-Thr-Ile-His-Pro-Phe

### Asp-Arg-Val-Tyr-Leu-His-Pro-Phe

### Asp-Arg-Val-Tyr-Ile-Arg-Pro-Phe

### Asp-Arg-Val-Tyr-Ile-His-Ala-Phe

### Asp-Arg-Val-Tyr-Ile-His-Pro-Tyr

### Pro-Arg-Val-Tyr-Ile-His-Pro-Phe

### Asp-Arg-Pro-Tyr-Ile-His-Pro-Phe

### Asp-Ar-Val-Tyr

### 2-Ile-His-Pro-Phe

### Asp-Arg-norLeu-Tyr-Ile-His-Pro-Phe

### Asp-Arg-Val-Tyr-norLeu-His-Pro-Phe

### Asp-Arg-Val-homoSer-Tyr-Ile-His-Pro-Phe

Other suitable ACE inhibitors for use in the present invention are disclosed in Oshima et al., “Peptide inhibitors of angiotensin I-converting enzyme in digests of gelatin by bacterial collagenase” (Biochem Biophys Acta 1979; 566: 128-137), and in Nakamura et al., “Purification and characterization of angiotensin I-converting enzyme inhibitors from a sour milk” (J Dairy Sci 1995; 78: 777-783) and Maruyama, S et al., “A peptide inhibitor of angiotensin I converting enzyme in the tryptic hydrolysate of casein”, Agric. Biol. Chem. 46 (5), 1393-1394 (1982).

In one preferred embodiment of the present invention, the ACE inhibitor for use in any of the methods, uses and compositions of the present invention is a non-thiol-containing ACE-inhibitor (i.e. it does not contain a thiol group).

In another preferred embodiment, the ACE inhibitor for use in any of the methods, uses and compositions of the present invention is a thiol-containing ACE-inhibitor (i.e. it comprises at least one thiol group).

In one preferred embodiment of the present invention, the ACE inhibitor is lipophilic, such as selected from quinapril, quinaprilat, trandolaprilat, trandolapril, moexipril, moexiprilat, fosinoprilat, fosinopril, benazeprilat, benazepril, enalaprilat or enalapril. More preferably, said ACE inhibitor is selected from quinaprilat, trandolaprilat, moexiprilat, fosinoprilat, benazeprilat or enalaprilat. In another preferred embodiment of the present invention, the ACE inhibitor is non-lipophilic, such as selected from the following: captopril, lisinopril, ramaprilat or ramapril.

In one preferred embodiment of the present invention, the ACE inhibitor binds to the zinc-binding ligand of the active site of ACE via a sulfhydryl group, such as captopril, zofenopril and/or alacepril. In another preferred embodiment of the present invention, said ACE inhibitor binds to the zinc-binding ligand of the active site of ACE via a phosphinyl group, such as fosinopril. In another preferred embodiment of the present invention, said ACE inhibitor binds to the zinc-binding ligand of the active site of ACE via a carboxyl group, such as ramipril or lisinopril.

In a preferred embodiment, the ACE inhibitors for use in the present invention are selected from captopril, enalaprilat, lisinopril, benazeprilat, fosinoprilat, moexiprilat, ramiprilat, trandolaprilat or quinaprilat. More preferably, said ACE inhibitor is ramiprilat or lisinopril. Alternatively, said ACE inhibitor is lisinopril or captopril. Most preferably, the ACE inhibitor is lisinopril.

### Cosmetic Composition

In one aspect of the present invention disclosed herein, a cosmetic composition is provided. Said cosmetic composition is suitable for use in any of the cosmetic methods or uses described herein. The cosmetic composition comprising at least one ACE inhibitor and/or angiotensin II receptor antagonist, or a cosmeceutically acceptable salt thereof. By “ACE inhibitor” is meant any substance capable of inhibiting, fully or partially, the biological functions of ACE. By angiotensin II receptor antagonist is meant any substance capable of antagonising, fully or partially, the biological functions of an angiotensin receptor. Preferably, said ACE inhibitor and/or angiotensin II receptor antagonist is selected from any of the groups described in the section above, entitled: “ACE inhibitors and angiotensin II receptor antagonists suitable for use in any of the methods, uses and compositions of the present invention”, or a cosmeceutically-acceptable salt thereof. In one embodiment of the present invention, said composition comprises more than one ACE inhibitor or angiotensin II receptor antagonist, or a cosmeceutical salt thereof.

Preferably, said ACE inhibitor or angiotensin II receptor antagonist is present in an concentration between about 0.01 mg/kg-100 mg/kg, such as 0.1 mg/kg-90 mg/kg, such as 0.5 mg/kg-75 mg/kg, such as 1 mg/kg-60 mg/kg, such as 2 mg/kg-45 mg/kg, such as 5 mg/kg-30 mg/kg, such as 10 mg/kg-15 mg/kg, such as 10 mg/kg-12 mg/kg. In another preferred embodiment, said ACE inhibitor or angiotensin II receptor antagonist is present in an amount between about 0.1 mg/kg-10 mg/kg.

In one preferred embodiment of the present invention, the cosmetic compositions according to the invention can take the form of any suitable cosmetic product. Preferably, the compositions take the form of a care, treatment, cleaning or protection product for the face or the body skin, including the scalp, such as a day and/or night and/or hydrating care composition for the face or the body; an anti-wrinkle or anti-ageing composition for the face; a composition for irritated skins; a make-up removing composition; a body milk, a sun protective, artificial sun tanning (self-tanning) or after-sun care composition; a sun protective cream or gel; a face skin, body or lip makeup product, such as a foundation cream, a tinted cream, a cheek or eye-lid makeup product, a free or compact powder, an anti eye-ring stick, a concealing stick, a lipstick or a lip care product. More preferably, said cosmetic composition exhibits the form of an anti-ageing composition.

The composition may also be applied to any part of the human body where skin improvement benefits are desired. More preferably, compositions of the present invention are used on areas exposed to the sun, such as the hands, scalp, face, lips arms, legs. In the most preferred embodiment of the present invention, the compositions are applied to the facial area.

### Cosmetic Method

In another aspect of the present invention, a cosmetic (i.e. non-therapeutic) method is provided for improving and/or maintaining the skin tone of an individual, said method comprising contacting the skin of said individual with the cosmetic composition described herein. Preferably, said method for improvement and/or maintenance of skin tone comprises prevention or reduction of skin ageing and/or wrinkling. By “skin tone” herein is meant any aspect of the skin, such as the texture, suppleness, moisture levels, radiance, smoothness and surface appearance, such as the presence of wrinkles and/or fine lines. By “maintaining the skin tone” is meant any process in which any cosmetically undesirable changes in the skin tone are lessened or even prevented. For example, in one embodiment the visible signs of fine lines on the skin are reduced. It should however be underlined that such aspects not relating to the skin layers themselves, i.e:
(i) hair growth (or lack of it)
(ii) discharge of sebum from the sebaceous glands and/or acne
(iii) growth of the nails
(iv) lymphatic drainage
(v) fatty mass
(vi) water retention and/or local oedemas
(vii) sodium imbalance and/or local oedemas
   are specifically excluded from the definition of “skin tone” as used herein. It is preferred that the cosmetic method provided herein is for reducing uneven collagen deposits present in aged and prematurely aged skin, thus evening out the skin texture.

The compositions of the invention can be applied to the skin on an as-needed basis, for example, they can be applied to the skin in the morning and/or in the evening, for instance every evening, and/or during the day. It is preferred that topical application be once a month to about 7 or 8 times daily, preferably from about 7 times a week to about 4 times a day, most preferably about once or twice a day. In another preferred embodiment, for an intensive treatment programme the compositions of the invention can be applied on a frequent basis throughout the day and/or night, such as 8 times daily.

To maintain the beneficial effects of the composition on the skin, it is preferred that the cosmetic method comprises repeatedly performing said contacting over an extended period of time, preferably over the lifetime of the user, equally preferably over a period from 4 weeks to twenty years, more preferably from about 6 months to about five years, resulting in the improvement and/or maintenance of an individual's skin tone. In another preferred embodiment of the present invention, the contacting is conducted at least once daily.

In one preferred embodiment of the present invention, the individual is a post-menopausal, female human being. In another, equally preferred embodiment, said individual is a pre-menopausal, female human being. In another, equally preferred embodiment, said individual is a male human being.

In one preferred embodiment, the cosmetic methods disclosed herein may further comprise contacting the skin with one or more other compound (such as co-formulated), as described in the “combinations” section below.

### Use of an ACE Inhibitor or Angiotensin II Receptor Antagoniser for the Preparation of a Medicament for the Treatment of Skin Ageing or Wrinkling

By “treatment” when discussing medical methods and practices herein, is meant one or more of prophylaxis, cure, lessening of pathological symptoms or other beneficial effect on an individual suffering from, or at risk of suffering from, a pathological condition, such as premature wrinkling.

By “premature wrinkling” is meant a greater density and/or depth of skin wrinkling and/or creasing of the skin than occurs for an average person of the same age, gender and racial background.

In one embodiment, the present invention relates to use of an ACE inhibitor and/or angiotensin II receptor antagonist for the preparation of a medicament for the treatment of skin ageing or wrinkling. Preferably, said skin ageing or wrinkling is considered premature as compared to normal skin ageing and wrinkling. In one preferred embodiment of the present invention, said skin ageing or wrinkling is caused by, or associated with, diabetes mellitus. Equally preferably, said skin ageing or wrinkling is caused by, or associated with, corticoid hormone hypersecretion. Equally preferably, said skin ageing or wrinkling is caused by, or associated with, syndrome X, Hutchinson-Gilford progeria, Werners syndrome or Kindler syndrome. Equally preferably, said skin ageing or wrinkling is caused by, or associated with, a vitamin deficiency, preferably vitamin C deficiency. Equally preferably, said skin ageing or wrinkling is caused by, or associated with, a medical treatment that adversely affects the skin collagen matrix, preferably administration of glucocorticoids and derivatives thereof, or, equally preferably, administration of vitamin D or derivative thereof. Equally preferably, said skin ageing or wrinkling is caused by skin photo-ageing processes. Equally preferably, said skin ageing or wrinkling is caused by, or associated with, radiation therapy.

Preferably, the medicament comprises an ACE inhibitor or angiotensin II receptor antagonist or a pharmaceutically acceptable salt thereof. More preferably, said ACE inhibitor or angiotensin II receptor antagonist is selected from the above section, entitled: “Ace inhibitors and angiotensin II receptor antagonists suitable for use in any of the methods, uses and compositions of the present invention”, or a pharmaceutically/cosmetically acceptable salt thereof.

It is preferred that said the medicament is administered in a concentration equivalent of from 0.01 mg/kg body weight to 100 mg/kg body weight, such as 0.1 mg/kg body weight to 90 mg/kg body weight, such as 0.5 mg/kg body weight to 75 mg/kg body weight such as 1 mg/kg body weight to 60 mg/kg body weight, such as 2 mg/kg-45 mg/kg body weight, such as 5 mg/kg body weight to 30 mg/kg body weight, such as 10 mg/kg body weight to 15 mg/kg body weight, such as 10 mg/kg body weight to 12 mg/kg body weight. In another preferred embodiment, said ACE inhibitor or angiotensin II receptor antagonist is present in an amount between about 0.1 mg/kg body weight-10 mg/kg body weight. It is further preferred that the compounds of the present invention in the medicament disclosed herein may be administered in combination with one or more other compounds, as described in the “combinations” section below.

In one preferred embodiment of the present invention, the individual is a post-menopausal, female human being. In another, equally preferred embodiment, said individual is a pre-menopausal, female human being. In another, equally preferred embodiment, said individual is a male human being. The compositions of the invention can be applied to the skin on an as-needed basis, for example, they can be applied to the skin in the morning and/or in the evening, and/or during the day. It is preferred that topical application be once a month to about 7 or 8 times daily, preferably from about 7 times a week to about 4 times a day, most preferably about twice a day. To maintain the beneficial effects of the medicament, it is preferred that said contacting is repeatedly performed over an extended period of time, preferably over the lifetime of the user, equally preferably for a period from 4 weeks to twenty years, more preferably from about 6 months to about five years, resulting in the treatment of skin ageing or wrinkling. In another preferred embodiment of the present invention, the contacting is conducted at least once daily.

In one preferred embodiment of the present invention, said medicament further comprises a pharmaceutically-acceptable topical carrier.

### Preferred Formulations of the Cosmetic and Pharmaceutical Compositions Disclosed Herein

The cosmetic or pharmaceutical compositions according to the invention can be formulated in any form acceptable for their use in cosmetics and/or in pharmacy. More preferably, the compositions of the present invention are formulated in any suitable manner for application to an individual's skin. Preferably, the composition is in a form appropriate for topical application, more preferably suitable for the application to the face, hands, bust or body, such as to the face or hands. The composition may also be used as a bust-firming composition or body-firming composition. It is preferred that said compositions are formulated as a lotion, face mask, skin patch, cream, ointment, water-based liquid, oil-based liquid, paste or sprayable liquid. More preferably, said composition is formulated as a cream or lotion. In another preferred embodiment, said composition is a face mask or skin patch.

In one preferred embodiment of the present invention, said formulation may contain ingredients such as absorbent particles (e.g. polymer beads or micelles) that provide sustained release of the compounds of the present invention to the skin. In one preferred embodiment, the formulations of the compositions of the present invention are hypo-allergenic, i.e. cause at the most a very low level of allergic reactions.

Compositions of the present invention can be directly applied, preferably to the skin, by any appropriate method, such as a spray bottle, a droplet bottle, a moisturized cotton ball or pad, suitable applicators such as paddles or strips, or by hands or fingers. In one preferred embodiment of the invention, compositions of the present invention may also be applied in a skin patch that incorporates cosmetic or pharmaceutical substances therein, such as those disclosed in French Patent Applications No. 2 512 651 and 2 538 247. In another, equally preferred embodiment of the present invention, compositions of the present invention may be applied in a skin “mask”, preferably in the form of a gels or paste, such as those skin masks disclosed in European Patent Application No. 0 063 875, Austrian Patent No. 206 114 and U.S. Pat. No. 5,026,552.

Another preferred form for topical delivery of the compounds of the present invention is a hot compress comprising a woven or non-woven fibrous wrap impregnated with one or more compounds of the present invention. It is preferred that prior to treatment the impregnated fibrous wrap is immersed in warm water to at least partially solubilize the active component and is wrapped around the area to be treated. Another preferred form of topical delivery is film-forming materials loaded with the compositions of the present invention. Such film-forming materials are, for example, disclosed in U.S. Pat. No. 4,623,539, which is incorporated herein by reference. Said film-forming polymers may include certain anionic, cationic and neutral polymers.

The compositions may also be packaged in the form of an aerosol composition containing a propellent agent under pressure.

It is preferred that the compositions of the present invention are combined with a cosmetically or pharmaceutically or dermatologically acceptable carrier. The total amount of the carrier preferably ranges from about 10 to about 99.9%, preferably from about 50 to about 90%, optimally from about 70 to about 85% by weight of the formulation.

### Cosmetic or Dermatological or Pharmaceutical Acceptable Carrier

“Cosmetic or dermatological or pharmaceutical acceptable carrier”, refers to a vehicle, for either cosmetic, dermatological or pharmaceutical use, which delivers the active components to their site of action and will not cause significant harm to the human or animal recipient. Any carrier selected for use in the therapeutic and cosmetic compositions should be pharmaceutically and/or cosmetically acceptable and appropriate for the form in which the composition will be used, e.g., cream, gel, milk, oil, lotion, face mask, skin patch, ointment, water-based liquid, oil-based liquid, paste, sprayable liquid and the like. Preferably, the carrier has an affinity for the skin, and/or is well tolerated and/or stable and/or it is used in an amount adequate to provide the desired consistency and ease of application.

The physiologically acceptable carrier in which the compounds according to the invention can be used, as well as the components thereof, their amount, the galenic form of the composition and its preparation mode, can be selected by the man of the art on the basis of its general knowledge depending on the type of the desired composition. Those skilled in the art will appreciate that a wide variety of pharmaceutically or cosmeceutically-acceptable carriers may be employed according to the present invention. Examples of such carriers are described in U.S. Pat. No. 4,877,805 and EPA Pub. No. 0586106A1.

In one embodiment of the present invention, said carrier may be a simple combination of a buffered solution of propylene glycol, and an acrylate gelation agent, or any of a wide variety of known or commercially available formulations for e.g. creams or lotions. More than one type of carrier may be used. In a preferred embodiment of the present invention, said carrier has been shown to have beneficial effects for wrinkle reduction. Thus, for example, the carrier may be that disclosed according to U.S. Pat. No. 5,885,596, hereby incorporated by reference.

For applying onto the skin, the composition can have the form in particular of an aqueous or an oily solution; of a dispersion of the lotion or serum type, of emulsions of liquid or semi-liquid consistency of the milk type obtained through dispersion of a fatty phase into an aqueous phase (O/W) or reversely (W/O); of suspensions or emulsions of a soft consistency of the cream type or aqueous or anhydrous gel type; of microcapsules or microparticles; of vesicular dispersions of the ionic and/or non ionic type. When the composition is in an aqueous form, in particular in an aqueous dispersion, emulsion or solution, it can comprise an aqueous phase, which may comprise water, flower water and/or mineral water. Said aqueous phase can additionally comprise alcohols such as C₁-C₆ monoalcohols and/or polyols such as glycerol, butyleneglycol, isoprene glycol, propyleneglycol, polyethyleneglycol. Ointments and creams may be formulated with an aqueous or oil base with the addition of suitable thickening or gelling agents. Lotions may be formulated with an aqueous or oily base. Powders may be formulated with the aid of any suitable powder base, such as talc, lactose, starch and the like. Ointments, pastes, creams and gels of the present invention may contain excipients, such as paraffins, starch, tragacanth, cellulose derivatives, polyethylene glycols, silicones, talc and zinc oxide.

Generally, the carrier can be anhydrous or aqueous. It can thus comprise an aqueous phase and/or a fatty phase. Thus, in one preferred embodiment of the present invention, the compositions comprise a fatty phase, in particular made of fatty bodies liquid at 25° C., such as oils from animal, vegetable, mineral or synthetic origin, either volatile or not, fatty bodies solid at 25° C. such as waxes from animal, vegetable, mineral or synthetic origin; of pasty fatty bodies; of gums; and the mixtures thereof. The volatile oils are generally oils having, at 25° C., a saturating vapor tension at least equal to 0.5 millibar (50 Pa). Fatty phase components include, but are not restricted to: cyclic volatile silicones having 3 to 8 silicon atoms, preferably 4 to 6, cyclocopolymers of the dimethylsiloxane/methylalkylsiloxane type, linear volatile silicones with 2 to 9 silicon atoms, hydrocarbon volatile oils, such as isoparaffins and, more particularly, isododecane and fluorinated oils, poly(C₁-C₂₀)alkylsiloxanes and, more particularly, those with trimethylsilyl end groups, amongst which linear polydimethylsiloxanes and alkylmethylpolysiloxanes such as cetyldimethicone (CTFA name), silicones modified by aliphatic and/or aromatic groups, optionally fluorinated, or by functional groups such as hydroxyl, thiol and/or amine groups, phenylated silicone oils, oils from animal, vegetable or mineral origin, in particular animal or plants oils made of esters of fatty acids and polyols, in particular liquid triglycerids, for example sunflower, corn, soya, marrow, grape seed, sesame, hazelnut, apricot, almond, or avocado oils; fish oils, glycerol tricaprocaprylate, or plant or animal oils having the formula R₁COOR₂, where R₁ represents the residue of a superior fatty acid having 7 to 19 carbon atoms and R₂ represents a branched hydrocarbon chain having 3 to 20 carbon atoms, for example Purcellin oil; paraffin oil, liquid paraffin, perhydrosqualene, wheatgerm, calophyllum, sesame, macadamia, grape seed, colza, copra, arachis, palm, castor, jojoba, olive or cereal germ oils; fatty acid esters; alcohols; acetylglycerides; octanoates, decanoates or ricinoleates from alcohols or polyalcohols; fatty acid triglycerids; glycerids; fluorinated and perfluorinated oils; silicone gums; waxes from animal, vegetable, mineral or synthetic origin, such as microcrystalline waxes, paraffin, petrolatum, liquid paraffin, ozokerite, Montan wax; beewax, lanolin, and the derivatives thereof; Candelilla, Ouricury and Japan waxes, cocobutter, cork fibre or sugar cane waxes; hydrogenated oils solid at 25° C., ozokerites, fatty esters and glycerides solid at 25° C.; polyethylene waxes and waxes obtained through Fischer-Tropsch synthesis; hydrogenated oils solid at 25° C.; lanolins; fatty esters solid at 25° C.; silicone waxes; fluorinated waxes.

Other suitable carriers for use with the present invention include, but are not limited to, water, mineral oil, ethylene glycol, propylene glycol, lanolin, glyceryl stearate, sorbitan stearate, isopropyl myristate, isopropyl palmitate, acetone, glycerol, phosphatidylcholine, sodium cholate, or ethanol.

When present, the amount of water in a composition may range anywhere from about 1 to about 99%, preferably from about 20 to about 90%, optimally between about 40 and 70% by weight.

The composition according to the invention can also comprise at least one co-emulsifier, which includes, but is not restricted to, oxyethylenated sorbitan monostearate, fatty alcohols such as stearyl alcohol or cetyl alcohol, or esters of fatty acids and polyols such as glyceryl stearate.

The compositions may also be combined with a skin penetration enhancer. The enhancers, helping to transport the active components through the normal intact skin, include, but are not limited to, liposomes, mixed lipid micelles, ethosomes, transfersomes, niosomes, ethanol, amides, ethers, glycols, hydrocarbon oils, sodium lauryl sulfate, oleic acid, hydroalcoholic solution, and soya phosphatidylcholine or their combinations. Other skin penetration enhancers includes use different pH values, co-solvents, surfactants, cyclodextrins, and iontophoresis. Said skin penetration enhancer is preferably in an amount ranging from 0.01 to 30% by weight based on the total weight of the composition. In one preferred embodiment, said skin penetration enhancer is a natural surfactants or an artificial surfactant such as isopropyl myristate.

Suitable solvents which can be used in the invention include lower alcohols, in particular, ethanol and isopropanol, and propylene glycol. Suitable hydrophilic gelling agents include carboxyvinyl polymers (carbomer), acrylic copolymers such as acrylate/alkylacrylate copolymers, polyacrylamides, polysaccharides such as hydroxypropylcellulose, natural gums and clays. Suitables lipophilic gelling agents include modified clays such as bentones, metal salts of fatty acids such as aluminum stearates, and hydrophobic silica, or alternatively ethylcellulose and polyethylene.

### Stabilization

It is preferred that the compositions of the present invention are stabilized. In general, stabilization methodologies and techniques that may be used in accordance with the present invention include any and all methods for the stabilization of chemical or biological material known to the art, including without limitation the addition of chemical agents, temperature modulation based methodologies; radiation based methodologies or combinations thereof. In preferred embodiments, small amounts of stabilizing chemical agents are mixed with the formulation comprising compositions of the present invention in order to achieve a stable preparation. These chemical agents preferably constitute less than approximately 10% (w/w), more preferably less than about 5% (w/w) and most preferably less than about 2.5% (w/w) of the formulation. Chemical agents that may be used in accordance with the present invention include inter alia preservative agents; acids; bases; salts; anti-oxidants; viscosity modifying agents; emulsifiers; gelling agents; and mixtures thereof.

In accordance with the present invention oxidative reactions may be prevented by the addition of anti-oxidants to the compounds of the present invention, for example butylated hydroxytoluened (BHT); butylated hydroxyanisol (BHA); methyl hydroxybenzoate, propyl hydroxybenzoate and benzalkonium chloride, ascorbic acid (vitamin C), tocopherol, tocopherol acetate, phytic acid, citric acid, pro-vitamin A, and mixtures thereof. More preferably, BHA and/or BHT are used.

The physical stability of the formulation of the compositions of the present invention may be further enhanced by the addition of emulsifying agents. Any emulsifying agent may be used. Examples of suitable emulsifying agents include, but are not restricted to, Arlacel, such as Alacel 165; or Glucamate.

Preservatives and/or antimicrobial actives are also suitable for use in combination with the compounds of the present invention, such as all antibiotics, antimicrobial agents and antimicrobial peptides. Antibiotics that may be used include inter alia dermatologically acceptable salts of tetracylin and tetracyclin derivatives, gentamycin, kanamycin, streptomycin, neomycin, capreomycin, lineomycin, paromomycin, tobramycin, erythromycin, triclosan, octopirox, parachlorometa xylenol nystatin, tolnafiate, miconazole hydrochloride, chlorhexidine gluconate, chlorhexidin hydrochloride, methanamine hippurate, methanamine mandelate, minocycline hydrochloride, clindamycin, cloecin, b-lactam derivatives such as aminopenicillin and mixtures thereof. Preferred compounds for use with the present invention are chlorhexidin gluconate and tricolosan. Anti microbial agents that may be used in accordance with the present invention include for example benzoyl peroxide and salicylic acid. Antimicrobial peptides useful herein are for example magainin, nicin and cecropin.

Other preservatives suitable for use in combination with the compounds of the present invention include, but are not restricted to, sodium metabisulfite, Glydant Plus, Phenonip, methylparaben, Germall 115, Germaben II, phytic acid, sodium lauryl sulfate (SLS), methyl hydroxybenzoate, propyl hydroxybenzoate, benzalkonium chloride, sodium lauryl ether sulfate (SLES), and mixtures thereof. In preferred embodiments non-formaldehyde donors such as Neolone, Kathon and Euxyl are used. alkyl esters of para-hydroxybenzoic acid, hydantoin derivatives, propionate salts, and a variety of quaternary ammonium compounds. Chemists are familiar with appropriate preservatives and routinely choose them to satisfy the preservative challenge test and to provide product stability. Particularly preferred preservatives are methyl paraben, imidazolidinyl urea, sodium dehydroxyacetate, propyl paraben and benzyl alcohol. The preservatives should be selected having regard for the use of the composition and possible incompatibilities between the preservatives and other ingredients in the composition. Preservatives are preferably employed in amounts ranging from about 0.01% to about 2% by weight of the composition.

### Viscosity Modifiers

Compositions of the invention can also include viscosity modifiers, preferably in amounts from about 0.01 to about 10% by weight of the composition. Viscosity modifiers such as cetyl alcohol, glycerol, polyethylene glycol (PEG), PEG-stearate, or Keltrol may also be used to enhance the stability of the formulation. Thickeners which may enhance the stability include gelling agents such as cellulose and derivatives, Carbopol and derivatives, carob, carregeenans and derivatives, xanthane gum, sclerane gum, long chain alkanolamides, bentone and derivatives, Kaolin USP, Veegum Ultra, Green Clay, Bentonite NFBC, magnesium aluminum silicate (Veegum®), guar gums (such as Jaguar HP-120®), xanthan gum, sodium carboxymethyl cellulose, hydroxyalkyl and alkyl celluloses, cross-linked acrylic acid polymers such as those sold by B.F. Goodrich under the Carbopol® trademark, and mixtures thereof. As known to those skilled in the art, the precise amount of thickeners can vary depending upon the consistency and thickness of the composition which is desired.

### Combinations

In one embodiment of the present invention, the compositions of the present invention may be administered in combination with other compounds, which may have a therapeutic, cosmetic or otherwise beneficial effect. By “in combination”, it is meant that said other compounds may administered before, concurrently with, or after, administration of the compositions of the present invention. Said other compounds may also be formulated with the ACE inhibitors and/angiotensin II antagonists of the present invention in the cosmetic compositions and/or medical compositions disclosed herein, in which case said other compounds, depending on their nature, may for example be introduced into the fatty phase, into the aqueous phase and/or into lipid spherules, of the compositions of the present invention. The nature and the amount of said other compounds can be selected one skilled in the art, based on common general knowledge, so as to obtain the desired presentation form for the composition. When said other compounds are added to the cosmetic and pharmaceutical compositions of the present invention, one skilled in the art could make sure to select suitable amounts of said other compounds, so that the advantageous properties of the composition according to the invention are not, or substantially not, altered by the contemplated addition.

Preferred compounds suitable for administration in combination with the compositions of the present invention comprise, but are not restricted to, hormones, plant and/or herbal extracts, moisturizers or humectants, emollients, fragrances, sun-screen actives, anti-wrinkle and/or anti-ageing actives, whitening and/or bleaching actives, sunless tanning actives, preservative and/or antimicrobial actives, anti-acne actives, anti-psoriasis actives, anti-eczema actives, anti-inflammatory actives, vitamin actives, proteins, peptides, amino acids, amino acid derivatives, insect repellants, fungicides, anti-viral agents, anti-cancer agents, anti-hemorrhoid compounds, anti-dandruff compounds, hair-growth stimulating compounds, hair-loss stimulating compounds, nucleic acids, chelating agents, pigments, lipids and/or inorganic salts.

In a preferred embodiment of the present invention, the compositions of the present invention are administered in combination with more than one other compounds, such as 2, 3, 4, 5, 6, 7, 8, 9, 10, 11, 12, 13, 14, 15, 16, 17, 18, 19, 20, 21, 22, 23, 24, 25, 26, 27, 28, 29 or 30 other compounds. In another preferred embodiment of the present invention, the compositions of the present invention are administered in combination with over 30 other compounds, such as 35-40, such as 40-45, such as 45-50, such as 50-100 other compounds.

### Hormones

Preferred examples of suitable other compounds comprise hormones, such as oestrogenic, progestative or androgenic hormones, including but not restricted to progesterone, testosterone, anhydrous oestradiol, broparestrol, oestrone, pregnenolone acetate, pregnenolone, 17-β-hydroxyprogesterone, testosterone propionate, androstenedione and androstanediols. Said hormones may be natural or synthetic.

### Plant and Herbal Extracts

Equally preferred other compounds suitable for use in combination with the compounds of the present invention include, but are not restricted to, plant or herbal extracts or chemically synthesised equivalents, such as extracts of Paraguay tea, Kola and Guarana, mate, marama bean, aloe vera; cryocytol; avocado; chamomile; echinacea; ginko biloba; ginseng; green tea; heather; jojoba; lavender; evening primrose oil, Marigold, Almond oil, safflower oil, jojoba oil, wheat germ oil, Horse-chestnut, Cucumber, Ivy, Bladderwort, Jodalga extract, lemon grass; licorice; mallow; oats; peppermint; St. John's wort; willow; wintergreen; wheat wild yam; Ubiquinone Q10, Retinoids, Alpha hydroxy acids (AHAs), Antocopherol and marine extracts, such as seaweed extract.

### Moisturizers and Humectants

Equally preferred compounds suitable for use in combination with the compounds of the present invention include, but are not limited to, one or more moisturizers. As used herein a “moisturizer” is an ingredient which promotes the retention of water to the surface area of the human body, including hair and skin. The term moisturizer as used herein includes both components which deliver water to the skin, also commonly referred to in the art as “humectant”, and components which prevent the loss of water from the skin, also commonly referred to in the art as “occlusive”. If present in the compositions of the present invention, said moisturizer will generally comprise from about 0.1% (w/v) to about 99% (w/v), more preferably from about 0.5% (w/v) to about 50% (w/v), and most preferably from about 1% (w/v) to about 40% (w/v) of the final composition.

Although the ingredients mentioned herein are generally defined as moisturizers they may also possess other properties such as emolliency or other conditioning properties. Moisturizers suitable for use in combination with the compounds of the present invention include, but are not limited to, polyhydroxy alcohols, including butylene glycol, hexylene glycol, propylene glycol, sorbitol and the like; lactic acid and lactate salts, such as sodium or ammonium salts; C₃ and C₆ diols and triols including hexylene glycol, 1,4 dihydroxyhexane, 1,2,6-hexane triol; aloe vera in any of its forms, for example aloe vera gel; sugars and starches; sugar and starch derivatives, for example alkoxylated glucose; hyaluronic acid; lactamide monoethanolamine; acetamide monoethanolamine; glycolic acid; alpha and beta hydroxy acids (e.g. lactic, glycolic salicylic acid); glycerine; pantheol; urea; vaseline; natural oils; oils and waxes (see: the emollients section herein) and mixtures thereof.

Humectants of the polyhydric alcohol-type may also be used in combination with the compositions of this invention. Possible roles of the humectant may be to aid in increasing the effectiveness of an emollient, reduce scaling, stimulate removal of built-up scale and improve skin feel. Typical polyhydric alcohols include polyalkylene glycols and more preferably alkylene polyols and their derivatives, including propylene glycol, dipropylene glycol, polypropylene glycol, polyethylene glycol and derivatives thereof, sorbitol, hydroxypropyl sorbitol, hexylene glycol, 1,3-butylene glycol, 1,2,6-hexanetriol, ethoxylated glycerol, propoxylated glycerol and mixtures thereof. For best results the humectant is preferably glycerol.

For improved lubricity, there may also be included one or more silicone oils or fluids which may be selected from a dimethyl polysiloxane, a methylphenyl polysiloxane and an alcohol-soluble silicone glycol copolymer. Preferred siloxanes include dimethyl polysiloxane (CTFA name: dimethicone), a polysiloxane end-blocked with trimethyl units and polydimethylcyclosiloxane, (CTFA name: cyclomethicone). The preferred siloxanes exhibit a viscosity from about 2 to 50 centistokes at 25° C.

### Emollients

Equally preferred compounds suitable for use in combination with the compounds of the present invention include, but are not limited to, one or more emollients. Emollients may be used, for example, to add or replace lipids and natural oils to the surface area of the human body. The term emollient as used herein is intended to include conventional lipids (for example, oils, waxes, lipids and other water insoluble components) and polar lipids (lipids which have been modified in order to increase water solubility typically through esterfication of a lipid to a hydrophylic moiety for example hydroxy groups, carbonyl groups and the like). Preferred emollients suitable for use in combination with the compounds of the present invention include, but are not limited to, those selected from the group consisting of natural oils and preferably plant-derived and essential oils, esters, silicone oils, polyunsaturated fatty acids (PUFAs), lanoline and its derivatives and petrochemicals.

Natural oils which may be used in combination with the present invention include, but are not restricted to, those obtained from sesame; soybean; apricot kernel; palm; peanut; safflower; coconut; olive; cocoa butter; palm kernel; shea butter; sunflower; almond; avocado; borage; carnauba; hazel nut; castor; cotton seed; evening primrose; orange roughy; rapeseed; rice bran; walnut; wheat germ; peach kernel; babassu; mango seed; black current seed; jojoba; macademia nut; sea buckthorn; sasquana; tsubaki; mallow; meadowfoam seed; coffee; emu; mink; grape seed; thistle; tea tree; pumpkin seed; kukui nut; and mixtures thereof. Esters which may be used in combination with the present invention include, but are not restricted to, C₈-C₃₀ alklyl esters of C₈-C₃₀ carboxylic acids; C₁-C₆ diol monoesters and diesters of C₈-C₃₀ carboxylic acids; C₁₀-C₂₀ alcohol monosorbitan esters, C₁₀-C₂₀ alcohol sorbitan di- and tri-esters; C₁₀-C₂₀ alcohol sucrose mono-, di-, and tri-esters and C₁₀-C₂₀ fatty alcohol esters of C₂-C₆ 2-hydroxy acids and mixtures thereof. Examples of these materials include isopropyl palmitate; isopropyl myristate; isopropyl isononate; C₁₂/C₁₄ benzoate ester (also known as Finesolve); sorbitan palmitate, sorbitan oleate; sucrose palmitate; sucrose oleate; isostearyl lactate; sorbitan laurate; lauryl pyrrolidone carboxylic acid; panthenyl triacetate; and mixtures thereof.

Other preferred emollients include silicone oils, including non-volatile and volatile silicones. Examples of preferred silicone oils suitable for use in combination with the compounds of the present invention include, but are not limited to, dimethicone; cyclomethycone; dimethycone-copolyol; aminofunctional silicones; phenyl modified silicones; alkyl modified silicones; dimethyl and diethyl polysiloxane; mixed C₁-C₃₀ alkyl polysiloxane; and mixtures thereof. Equally preferred silicones are described in U.S. Pat. No. 5,011,681 to Ciotti et al., incorporated by reference herein. Equally preferred emolliants suitable for use in combination with the compounds of the present invention include, but are not limited to, lanoline and lanoline derivatives for example lanoline esters. Petrochemicals suitable for use as emolliants in combination with the compounds of the present invention include, but are not limited to, mineral oil; petrolatum; isohexdecane; permethyl 101; isododecanol; C₁₁-C₁₂ Isoparrafin, also known as Isopar H.

Waxes suitable for use in combination with the compounds of the present invention include, but are not limited to, animal waxes such as beeswax; plant waxes such as carnauba wax, candelilla wax, ouricurry wax, Japan wax or waxes from cork fibres or sugar cane, mineral waxes, for example paraffin wax, lignite wax, microcrystalline waxes or ozokerites and synthetic waxes.

Other emollients suitable for use in combination with the compositions of the present invention include, but are not restricted to, hydrocarbon oils and waxes, such as mineral oil, petrolatum, paraffin, ceresin, ozokerite, microcrystalline wax, polyethylene, and perhydrosqualene; triglyceride esters such as vegetable and animal fats and oils, such as castor oil, cocoa butter, safflower oil, cottonseed oil, corn oil, cod liver oil, almond oil, avocado oil, sesame oil, squalene, and maleated soybean oil; acetoglyceride esters, such as acetylated monoglycerides; ethoxylated glycerides, such as ethoxylated glyceryl monostedrate; alkyl esters of fatty acids having 10 to 22 carbon atoms, such as methyl, isopropyl, and butyl esters of fatty acids, such as hexyl laurate, isohexyl laurate, isohexyl palmitate, isopropyl palmitate, decyl oleate, isodecyl oleate, hexadecyl stearate, decyl stearate, isopropyl isostearate, diisopropyl adipate, diisohexyl adipate, dihexyldecyl adipate, diisopropyl sebacate, lauryl lactate, myristyl lactate, and cetyl lactate; alkenyl esters of fatty acids having 10 to 22 carbon atoms, such as oleyl myristate, oleyl stearate, and oleyl oleate; fatty acids having 10 to 22 carbon atoms, such as pelargonic, lauric, myristic, palmitic, stearic, isostearic, hydroxystearic, oleic, linoleic, ricinoleic, arachidic, behenic, and erucic acids; fatty alcohols having 10 to 22 carbon atoms, such as lauryl, myristyl, cetyl, hexadecyl, stearyl, isostearyl, hydroxystearyl, oleyl, ricinoleyl, behenyl, erucyl, and 2-octyl dodecanyl alcohols; fatty alcohol ethers, such as ethoxylated fatty alcohols of 10 to 22 carbon atoms, such as the lauryl, cetyl, stearyl, isostearyl, oleyl, and cholesterol alcohols, having attached thereto from 1 to 50 ethylene oxide groups or 1 to 50 propylene oxide groups; ether-esters such as fatty acid esters of ethoxylated fatty alcohols; lanolin and derivatives, such as lanolin, lanolin oil, lanolin wax, lanolin alcohols, lanolin fatty acids, isopropyl lanolate, ethoxylated lanolin, ethoxylated lanolin alcohols, ethoxylated cholesterol, propoxylated lanolin alcohols, acetylated lanolin alcohols, lanolin alcohols linoleate, lanolin alcohols ricinoleate, acetate of lanolin alcohols ricinoleate, acetate of ethoxylated alcohols-esters, hydrogenolysis of lanolin, ethoxylated hydrogenated lanolin, ethoxylated sorbitol lanolin, and liquid and semisolid lanolin absorption bases; polyhydric alcohol esters such as ethylene glycol mono and di-fatty acid esters, diethylene glycol mono- and di-fatty acid esters, polyethylene glycol (200-6000) mono- and di-fatty acid esters, propylene glycol mono- and di-fatty acid esters, polypropylene glycol 2000 mono-oleate, polypropylene glycol 2000 monostearate, ethoxylated propylene glycol monostearate, glyceryl mono- and di-fatty acid esters, polyglycerol poly-fatty esters, ethoxylated glyceryl monostearate, 1,3-butylene glycol monostearate, 1,3-butylene glycol distearate, polyoxyethylene polyol fatty acid esters, sorbitan fatty acid esters, and polyoxy-ethylene sorbitan fatty acid esters; wax esters such as beeswax, spermaceti, myristyl myristate, stearyl stearate; beeswax derivatives, such as polyoxyethylene sorbitol beeswax; vegetable waxes including carnauba and candelilla waxes; phospholipids such as lecithin and derivatives; sterols, cholesterol and cholesterol fatty acid esters; amides such as fatty acid amides, ethoxylated fatty acid amides and solid fatty acid alkanolamides.

When formulated with the compositions of the present invention, emollients preferably range from about 0.5 to about 80% by weight of the total composition. Preferably the amounts of these emollients will range from about 1 to about 25%, optimally between about 5 and 15% by weight. Equally preferably, said emollients typically comprise between from about 0.01% to about 25%, preferably from about 0.05% to about 15% and more preferably from about 0.1% to about 10% w/v of the total formulation.

It is noted that although the ingredients mentioned herein are generally defined as emollients they may also possess other properties such as moisturization or other advantageous properties.

### Fragrances

Equally preferred compounds suitable for use in combination with the compounds of the present invention include, but are not limited to, fragrances, preferably one or more fragrances without inherent adverse effects. If comprised in the pharmaceutical or cosmetic compositions of the present invention, said fragrance preferably comprises between about 0.0001% (v/v) and about 25% (v/v) of the final composition, more preferably between about 0.001% (v/v) and 10% (v/v) and most preferably between 0.01% (v/v) and 5% (v/v) of the final composition. For the purpose of the present application the term “fragrance” is meant to encompass any component reacting with the human olfactory sites and imparting a pleasurable odor, essence or scent.

Fragrances that may be used in accordance with the present invention include any synthetic as well as natural fragrance and mixtures thereof. Typically a multiplicity of fragrances are used to achieve the desired effect. Those of skill in the art further recognize the terms “top note” (i.e. fragrances having a high vapor pressure), “middle note” (i.e. fragrance having a medium vapor pressure) and “base note” (i.e. fragrances having a low vapor pressure). Recognizing that categorization within these classes may depend to some extent on the fragrance formulator, the fragrances used in combination with the present invention may comprise any top note, middle note and base note fragrance.

A further way of classifying fragrances is in accordance with generally recognized scents they produce. Descriptions used by those skilled in the art of fragrances are inter alia “rose”, “floral”, “green”, “citrus”, “spicy”, “honey”, “musk”, “herbal”, “jasmin”, “lilac”, “lily of the valley”, “orange”, “peach”, “oriental”, “watermelon” “chypre” and “lemon”, “woody”, “fruity” all of which fragrances thus classified may used in combination with the present invention. Preferred fragrances suitable for use in combination with the compounds of the present invention include, but are not limited to, linear and cyclic alkenes (i.e. terpenes); primary, secondary and tertiary alcohols; ethers; esters; ketones; nitrites; and saturated and unsaturated aldehydes; or mixtures thereof.

Equally preferred fragrances suitable for use in combination with the present invention include synthetic fragrances, such as one or more of acetanisole; acetophenone; acetyl cedrene; methyl nonyl acetaldehyde; musk anbrette; heliotropin; citronellol; sandella; methoxycitranellal; hydroxycitranellal; phenyl ethyl acetate; phenylethylisobutarate; gamma methyl ionone; geraniol; anethole; benzaldehyde; benzyl acetate; benzyl salicate; linalool; cinnamic alcohol; phenyl acetaldehyde; amyl cinnamic aldehyde; caphore; p-tertiairy butyl cyclohexyl acetate; citral; cinnamyl acetate; citral diethyl acetal; coumarin; ethylene brasslate; eugenol; 1-menthol; and vanillin.

Equally preferred fragrances suitable for use in combination with the present invention include natural fragrances, such as one or more of lavandin; heliotropin; sandlewood oil; oak moss; pathouly; ambergris tincture; ambrette seed absolute; angelic root oil; bergamont oil; benzoin Siam resin; buchu leaf oil; cassia oil; cedar-wood oil; cassia oil; castoreum; civet absolute; chamomile oil; geranium oil; lemon oil; lavender oil and Ylang Ylang oil.

Equally preferred fragrances suitable for use in combination with the present invention include all the fragrances disclosed in “Perfume and Flavor Chemicals”, Vols. I and II; Steffen Arctander Allured Pub. Co. (1994) and “Perfumes: Art, Science and Technology”; Muller, P. M. and Lamparsky, D., Blackie Academic and Professional (1994) both incorporated herein by reference.

### Sunscreen Actives

Equally preferred compounds suitable for use in combination with the present invention include one or more sunscreen active. The term “sunscreen” is used to denote ultraviolet ray-blocking compounds inhibiting absorption within the wavelength region between 290 and 420 nm. These compounds may either be organic or inorganic. Typical inorganic sunscreens include titanium dioxide, zinc oxide, iron oxide and combinations thereof. Most preferred is titanium dioxide, especially having an average particle size no higher than 700 nm, preferably no higher than 200 nm, optimally less than 35 nm. Organic sunscreens may be classified into five groups based upon their chemical structures: para-amino benzoates; salicylates; cinnamates; benzophenones; coumarins; azoles and miscellaneous chemicals including menthyl anthralinate. Also polymeric particles may be useful such as polyethylene and polyamides. If incorporated into the compositions of the present invention, said organic sunscreen compounds preferably range in amount from about 0.1 to 25%, optimally from about 1 to 15%, most preferably from about 5 to 10% by weight. A wide variety of sunscreen actives are useful herein.

The exact amount and type of sunscreen that is used depends on the level of photoprotection that is desired. Generally, any agent offering protection against ultraviolet radiation by absorbing, scattering or reflecting the ultraviolet radiation may be used herein. The sunscreen agents may offer protection against one or more of the following forms of sunlight radiation UVA, UVB, UVC, visible light and infrared radiation. Generally the sunprotection factor (SPF) in the final formulation varies between 2 and 30, although products with SPFs up to 100 may be formulated. The sunscreen used herein may offer chemical or physical photoprotection. UVA and UVB blocking agents, such as those disclosed according to the U.S. Pat. No. 6,130,254 patent, may be included to provide a composition effective at preventing, minimizing or avoiding photoaging.

Equally preferred sunscreens suitable for use in combination with the compounds of the present invention include those selected from the group comprising amino benzoic acid and derivatives, such as para-amino benzoic acid (PABA), glyceryl-PABA (Lisadimate), Padimate O, Roxadimate; anthrinalates, including methylanthrynilate; benzophenones, including dioxybenzone, oxybenzone and sulisobenzone, 3-benzophenone (Uvinul M40) 4-N,N-dimethylaminobenzoic acid ester with 2,4-dihydroxybenzophenone; camphor derivatives including 3-(4-methylbenzylidene) camphor, 3-benzylidene camphor; cinnamates including DEA-p-methoxycinnamate, ethyl-hexyl p-methoxy cinnamate, octocrylene, octyl methoxy cinnamate (Parasol MCX); dibenzoyl methanes including butylmethoxydibenzoylmethane (Parasol 1789), salicylates including, homomethyl salicylate, octyl salicylate, trolamine methyl salicylate; metal oxides including titanium dioxide, zinc oxide and iron oxide; 2-phenylbenzimidazole-5-sulfonic acid; 4,4-methoxy-t-butyldibenzoylmethane; and mixtures thereof.

Further non-limiting examples of sunscreens useful in combination with the present invention are described in U.S. Pat. No. 5,087,445 to Haffey et al., U.S. Pat. No. 5,073,372 to Turner et al. and U.S. Pat. No. 5,160,731 to Sabatelli et al., all of which are incorporated herein by reference in their entirety.

### Anti-Wrinkle and Anti-Ageing Actives

Equally preferred compounds suitable for use in combination with the present invention include anti-wrinkle and anti-aging actives. These agents include without limitation hydroxy acids including C₂-C₃₀ alpha-hydroxy acids such as glycolic acid, lactic acid, 2-hydroxy butanoic acid, malic acid, citric acid tartaric acid, decorin-synthesis enhancers, retinoids which include retinol and its esters, retinal, retinoic acid and its derivatives, retinoids, and in particular those described in documents FR 2,570,377, EP 0 199 636, EP 0 325 540 and EP 0 402 072, alpha-hydroxy acids such as glycolic, lactic, malic, citric, tartaric or mandelic acid), beta-hydroxy acids such as salicylic acid and its derivatives, in particular its alkyl derivatives, alpha-keto acids, beta-keto acids, peroxides such as benzoyl peroxide, vitamins, in particular vitamins E and F. anti-free-radical active agents such as superoxide dismutase, selenium, zinc, beta-carotenes, tensioning polymers of natural or synthetic origin, collagen-synthesis enhancers, matrix metalloproteinases (MMP) inhibitors, antioxidants, collagen modulators, alpha-hydroxyethanoic acid, hydroxycaprylic acid and the like; alpha-hydroxycarboxylic acids or salts thereof, beta-hydroxycarboxylic acid or salts thereof, ceramides, polyhydroxy acids including gluconolactone (G4), gamma-linolenic acid, fruit acids, sugar cane extract and glycomer in cross-linked alpha nutrium; skin peel agents such as phenol, phytic acid and acetic acid.

Whitening and/or Bleaching Actives

Equally preferred compounds suitable for use in combination with the compounds of the present invention include whitening and/or bleaching actives, which include hydroquinone and derivatives, kojic acid, lactic acid, niacinamide, ascorbyl acid and derivatives such as magnesium ascorbyl phosphate, arbutin, and licorice root. If incorporated directly into the cosmetic or pharmaceutical compositions of the present invention, said whitening or bleaching active is preferably present in an amount ranging from 0.001 to 10% by weight.

### Sunless Tanning Actives

Equally preferred compounds suitable for use in combination with the compounds of the present invention include sunless tanning actives, such as dihydroxyacetone (DHA); glyceryl aldehyde; tyrosine and tyrosine derivatives such as malyltyrosine, tyrosine glucosinate, and ethyl tyrosine; phospho-DOPA, indoles and derivatives; and mixtures thereof, possibly in combination with amines and/or amino acids. If incorporated directly into the cosmetic or pharmaceutical compositions of the present invention, said sunless tanning active is preferably present in an amount ranging from 1% to 30% by weight.

### Anti-Acne Actives

Equally preferred compounds suitable for use in combination with the compounds of the present invention include one or more anti-acne actives, including, but not restricted to, keratolytic agents including lactic acid, pyruvic acid, salicylic acids, urea and N-acetylcysteine; retinoids, and retinoid analogs such as tretinoin, cis and trans retinoic acid, retinol and retinol palmitate, isotretinoin-13-cis-retinoic acid; antibiotics and antimicrobial agents such as tetracycline, erythromycin, minocycline, clindamycin, trimethoprim-sulphamethazole and anti-microbial peptides (nicin, for example); steroids, such as hydrocortisone; gamma-linolenic acid and mixtures thereof. Further anti-acne actives that may be used include without limitation benzoyl peroxide; salicylic acid, alpha and beta hydroxy acids; sulfacteamide and sulfur and derivatives and mixtures thereof. Preferably used herein are salicylic acid, benzoyl peroxide and retinoids. If incorporated directly into the cosmetic or pharmaceutical compositions of the present invention, said anti-acne active is preferably present in an amount ranging 0.1 to 30% by weight of the composition.

### Anti-Psoriasis Actives

Equally preferred compounds suitable for use in combination with the compounds of the present invention include one or more anti-psoriasis actives, including but not restricted to salicylic acid; mometasone furoate; steroids including corticosteroids such as cortisone and oluxclobetasol propionate; 5-fluorouracil; epinephrine; anthralin; vitamin D3 analogs, such as calcipotriene; methotrexate; masprocol; trimethaxate gluconate; retinoids; cyclosporin; paclitaxel; 5-amino levulinic acid; berbergasol; tin-ethyl etio purpurin; benzoporphyrin derivatives; antibodies, such as ABX-IL8 antibody, CD11a monoclonal antibody and ICM3 monoclonal antibody; enzyme inhibitors, including tryptase inhibitor and phospholipase A-2 inhibitors; angiogenesis blocking agents; T-cell blocking agents and mixtures thereof.

### Anti-Eczema Actives

Equally preferred compounds suitable for use in combination with the compounds of the present invention include one or more of anti-eczema actives including, but not restricted to, urea, evening primrose oil, plant extracts, hydrocortisone, an immunomodulator, tar and/or fatty acids obtained from banana.

### Anesthetic Actives

Equally preferred compounds suitable for use in combination with the compounds of the present invention include, but are not restricted to, one or more anesthetic actives, such as tetracaine, lidocaine, editocaine, bupivacaine or pramoxine.

### Anti-Inflammatory Actives

Equally preferred compounds suitable for use in combination with the compounds of the present invention include one or more anti-inflammatory actives, such as steroidal actives such as hydrocortisone, or non-steroidal actives including propionic derivatives, acetic acid derivatives, biphenylcarboxylic acid derivatives, fenamic acid derivatives, and oxicams; or acetominaphen, oxaprozin, pranoprofen, benoxaprofen, bucloxic acid or elocon.

### Vitamin Actives

Equally preferred compounds suitable for use in combination with the compounds of the present invention include one or more of vitamin actives, including but not restricted to vitamin A and derivatives, including retinoic acid, retinyl aldehyde, retin A, retinyl palmitate, adapalene, and beta-carotene; vitamin B (panthenol, provitamin B5, panthenic acid, vitamin B complex factor); vitamin C (ascorbic acid and salts thereof) and derivatives such as ascorbyl palmitate; vitamin D including calcipotriene (a vitamin D3 analog) vitamin E including its individual constituents alpha-, beta-, gamma-, delta-tocopherol and cotrienols and mixtures thereof and vitamin E derivatives including vitamin E palmitate, vitamin E linolate and vitamin E acetate; vitamin K and derivatives; vitamin Q (ubiquinone) and mixtures thereof.

### Proteins and Peptides

Equally preferred compounds suitable for use in combination with the compounds of the present invention include one or more protein or peptide. Proteins and/or peptides may be formulated in any desired manner for combination with the compounds of the present invention, however in one preferred embodiment, said proteins and/or peptides are recombinant. Proteins and/or peptides which may be used in combination with the compounds of the present invention include, but are not restricted to, enzymes such as proteases (e.g. bromelain, papain, collagenase, elastase), lipases (e.g. phospholipase C), esterases, glucosidases, exfoliating enzymes; antibodies and antibody derived actives, such monoclonal antibodies, polyclonal antibodies, single chain antibodies and the like; reductases; oxidases; peptide hormones; natural structural skin proteins, such as elastin, collagen, reticulin and the like; growth factors such as platelet derived growth factor (PDGF) and epidermis derived growth factor (EGF); anti-oxidants such as superoxide dismutase, catalase and glutathione; free-radical scavenging proteins; DNA-repair enzymes, for example T4 endonuclease 5 and P53; antimicrobial peptides, such as magainin and cecropin; a milk protein; a silk protein or peptide; and any active fragments or derivatives of the above-mentioned proteins and peptides.

### Other Preferred Compounds

Equally preferred compounds suitable for use in combination with the compounds of the present invention include one or more of: an amino acid and amino acid derivative; an insect repellant; a fungicide (such as ketoconazole); an anti-viral agent (such as acyclovir); an anti-cancer agent; an anti-hemorrhoid compound; an anti-dandruff compound; a hair-growth stimulating compound; a hair loss stimulating compound; a nucleic acid (which may be natural or non-natural); an anti-wart agent (such as podophyllotoxin); chelating agents (capable of binding metal ions) such as tartaric acid, EDTA, citric acid, alkali metal citrates, pyrophosphate salts or anionic polymeric polycarboxylates; pigments, which may be white or coloured, inorganic or organic and/or paerlescent.

Preferred pigments comprise, but are not restricted to, titanium dioxide, zinc oxide, zirconium dioxide, black, yellow, red and brown iron oxides, cerium dioxide, chro—

### EXAMPLES

### Example 1

### Preparation of a Suitable Cream Base (O/W) for Use with the ACE Inhibitors and/or Angiotensin II Receptor Antagonists of the Present Invention

### Ingredients

| | | |
|---|---|---|
| Component | % | Function |

I.

| | | |
|---|---|---|
| Emulgade ® SE | 4.0 | O/W cream base |
| Glyceryl Stearate (and) Ceteareth-20 | | SE |
| (and) Ceteareth-12 (and) Cetearyl | | |
| Alcohol (and) Cetyl Palmitate | | |
| Cutina ® MD | 1.0 | Consistency |
| Glyceryl Stearate | | giving factor |
| Lanette ® O | 1.0 | Consistency |
| Cetearyl Alcohol | | giving factor |
| Baysilon M 350 (Bayer) | 0.5 | Defoamer |
| Dimethicone | | |
| Cetiol ® PGL | 7.0 | Emollient |
| Hexyldecanol (and) Hexyldecyl Laurate | | |
| Myritol ® 312 | 3.0 | Emollient |
| Caprylic/Capric Triglyceride | | |
| Cetiol ® OE | 4.0 | Emollient |
| Dicaprylyl Ether | | |
| Copherol ® 1250 | 0.5 | active ingredient |
| Tocopheryl Acetate | | |

II.

| | | |
|---|---|---|
| D-Panthenol (BASF) | 1.0 | active ingredient |
| Glycerin 86% | 5.0 | Moisturiser |
| Aqua | 71.5 | |

III.

| | | |
|---|---|---|
| Carbopol 980 (Goodrich) | 0.2 | Stabiliser |
| Carbomer | | |
| Cetiol ® PGL | 1.0 | Emollient |
| Hexyldecanol (and) Hexyldecyl Laurate | | |

IV.

| | | |
|---|---|---|
| KOH, 20% | 0.3 | Neutraliser |
| Perfume/preservative | n.B./q.s. | |
| Viscosity Brookfield, mPas | 100.000 | |
| RVF, 23° C., Spindel/spindle TE, | | |
| 4 UpM/rpm, Helipath | | |

### Preparation

1. Melt the components listed under I at 80-85° C. and stir until a homogeneous mixture results.

2. Heat the components listed under II to 80-85° C. and add to phase I with stirring/homogenizing. Add phase III (Carbopol mixed with oil) into the hot emulsion and homogenise immediately by means of a suitable dispersion unit (Ultra Turrax). Allow the emulsion to cool with stirring in such a way that it remains in continual motion. Avoid the incorporation of air. Add the single components listed under IV at 40° C. Allow to cool to 30° C.

(All products in the text marked with an ® are trademarks of the Cognis group.)

### Example 2

### Preparation of a Suitable Cream for Topical Application of an ACE Inhibitor

The cream base described in Example 1 may be formulated with any ACE inhibitor. For example, a lisinopril cream may be formulated by adding 10 mg/kg Lisinopril to the cream base. Another example may be by adding 10 mg/kg Ramiprilat to the cream base.

### Example 3

### Topical Application of ACE-Inhibitors

Long term exposure to ultraviolet irradiation causes premature skin ageing (photoageing) characterized by wrinkles and loss of skin tone. Photoaged skin displays prominent alterations in the collagenous extracellular matrix of connective tissue. A model for inducing a well-defined photoageing process is by high voltage irradiation to a well-defined region of the skin of mice. The role of topical application of Angiotensin Converting Enzyme (ACE)-inhibition for attenuating collagen damage in photoageing after irradiation and the formation of wrinkles will be investigated.

A study will be undertaken to test this hypothesis in a mice model using single dose irradiation to the thorax region—a well-defined region on the mice—and using skin fibrosis as the primary endpoint as evaluated by immunohistochemical measurement and in the local formation of procollagen type 1 and 3 and YKL-40.

Single dose high voltage irradiation to the thoracic region in C57bl/6J mice will be used as a model for photoageing in a well-defined skin area. The mice will be treated with 12, 15, 18 Gy in two groups. One group will be treated with a cream containing Ramiprilat once daily 24 hours after irradiation and in the other group the mice will be treated with the same cream without Ramiprilat. The primary endpoint will be the induction of skin fibrosis; the secondary endpoint will be the formation of mediators of skin fibrosis as procollagen type I and III and YKL-40.

### MATERIALS AND METHODS

### Mice

C57/BL/6 mice are chosen for this study because of their well characterized tolerance to irradiation. We have previously irradiated about 500 (12, 15 and 18 Gy all done twice with 60 mice in each trial) this way and they are stored in a refrigerator. We will use female mice. The mice will be obtained from Bomholtgaard (Denmark) 2 weeks before irradiation. The mice will be housed at 22 degrees, 8-10 mice in a cage. They will be fed on a conventional diet (Altomin Rat 13/24) and given slightly acidified water ad libitum. Mice weighing between 18 g and 22 g will be used for the investigation. After irradiation the mice will bee randomized to receive topical application of a cream with ramiprilat starting 24 hours after irradiation or topical application of the same cream without ramiprilat (placebo). Only the chief laboratory technician will know the randomization code. The identity of all mice will be secured by ear markings.

All mice will be treated according to the guidelines from the department of justice regarding laboratory animals.

### Irradiation

Immediately before irradiation all mice will be anesthetized with an intraperitoneal injection of a combination of fentanyl/fluanison (Hypnorm, Janssen-Cilag, UK) and midazolam (Dormicum, Hoffmann-La Roche, Switzerland). In the first studies they will be given either 12 Gy, 15 Gy or 18 Gy to the thorax. On each dose level 54 to 60 mice will be irradiated. The mice will be plastered to a Perspex plate with ordinary tape 6-7 at a time. The Perspex plate will be provided with parallel lines, one cm apart to help precise fixation of the mice. Irradiation will be performed using a Philips 4 MV linear accelerator.

The dose rate will be approximately 3 Gy/min. The field will be 20 mm wide. Port films will be taken to ensure that both lungs of the mice were entirely included in the field. During irradiation the mice will be covered with a 3 mm thick layer of tissue equivalent material (“superflab”) to ensure a homogeneous dose distribution in the skin. After a medium of 90 days the mice will be sacrified and their skin examined immunohistochemically and the amount of procollagen type I and III and YKL-40 was measured in the skin. The amount of wrinkles will be measured with a video camera.

### Statistics

The primary endpoint for the irradiation studies will be development of skin fibrosis. As secondary endpoint we will use the amount of procollagen type I and III and YKL-40 in the skin. The immunohistochemical amount of skin fibrosis will be graded in 4 levels and the median for two groups of mice (treated and placebo) will be compared with a Wilcoxon test for non-parametric data. For the concentrations of procollagen type II and II and YKL-40 a simple comparison of means by a t-test will be performed.

1. A cosmetic method for improving and/or maintaining the skin tone of an individual, said method comprising contacting the skin of said individual with a composition comprising at least one ACE inhibitor and/or angiotensin II receptor antagonist, or a cosmeceutically acceptable salt thereof.

2. The cosmetic method according to the preceding claim, wherein said ACE inhibitor and/or angiotensin II receptor antagonist is selected from Alacepril, Delapril Benazepril, Cilazapril, Captopril, Enlapril, Fosinopril, Lisinopril, Moexipril, Perindopril, Ramipril, Quinapril, Trandolapril, Imidapril, Isradipin, perindopril, spirapril, temocapril, Enalapril, losartan (Cozaar), valsartan (Diovan), irbesartan (Avapro), candesartan (Atacand), telmisartan (Micardis), eprosartan, tasosartan, zolarsartan, Zofenapril, Isradipin and Candesartancilexetil, or a cosmeceutically acceptable salt thereof.

3. The cosmetic method according to any of the preceding claims, wherein said ACE inhibitor and/or angiotensin II receptor antagonist is selected from Alacepril, Delapril, Cilazapril, Benazepril, Captopril, Enlapril, Fosinopril, Lisinopril, Moexipril, Perindopril, Ramipril, Quinapril, Trandolapril, Imidapril, Isradipin, perindopril, spirapril, temocapril, Enalapril, Zofenapril, or a cosmeceutically acceptable salt thereof.

4. The cosmetic method according to any of the preceding claims, wherein said ACE inhibitor or angiotensin II receptor antagonist is selected from losartan (Cozaar), valsartan (Diovan), irbesartan (Avapro), candesartan (Atacand), telmisartan (Micardis), eprosartan, tasosartan, zolarsartan, Isradipin, Candesartancilexetil and/or olmesartan medoxomil, or a cosmeceutically acceptable salt thereof.

5. The cosmetic method according to any of the preceding claims, wherein said ACE inhibitor is lisinopril, or a cosmeceutically acceptable salt thereof.

6. The cosmetic method according to any of the preceding claims, wherein said composition comprises at least two ACE inhibitor(s) and/or angiotensin II receptor antagonist(s).

7. The cosmetic method according to any of the preceding claims, wherein said ACE inhibitor or angiotensin II receptor antagonist is present in said compositions in an amount between about 0.01-100 mg/kg.

8. The cosmetic method according to any of the preceding claims, wherein said ACE inhibitor or angiotensin II receptor antagonist is present in said composition in an amount between about 0.1-10 mg/kg.

9. The cosmetic method according to any of the preceding claims, wherein said composition further comprises a cosmeceutically-acceptable topical carrier.

10. The cosmetic method according to any of the preceding claims, wherein said cosmetic method further comprises one or more of a hormone, plant and/or herbal extracts, moisturizers or humectants, emollients, fragrances, sun-screen actives, anti-wrinkle and/or anti-ageing actives, whitening and/or bleaching actives, sunless tanning actives, preservative and/or antimicrobial actives, anti-acne actives, anti-psoriasis actives, anti-eczema actives, anti-inflammatory actives, vitamin actives, proteins, peptides, amino acids, amino acid derivatives, insect repellants, fungicides, anti-viral agents, anti-cancer agents, anti-hemorrhoid compounds, anti-dandruff compounds, hair-growth stimulating compounds, hair-loss stimulating compounds, nucleic acids, chelating agents, pigments, lipids and/or inorganic salts.

11. The cosmetic method according to any of the preceding claims, wherein said composition further comprises one or more of a hydroxy acid, lactic acid, 2-hydroxy butanoic acid, malic acid, citric acid tartaric acid, decorin-synthesis enhancers, retinoids which include retinol and its esters, retinal, retinoic acid and its derivatives, retinoids, an alpha-hydroxy acid, a beta-hydroxy acid, an alpha-keto acids, a beta-keto acid, a peroxide, a vitamin, an anti-free-radical active agents, selenium, zinc, a beta-carotene, tensioning polymers of natural or synthetic origin, collagen-synthesis enhancers, a matrix metalloproteinase inhibitor, an antioxidant, a collagen modulator, an alpha-hydroxyethanoic acid, a hydroxycaprylic acid; an alpha-hydroxycarboxylic acid, beta-hydroxycarboxylic acid, a ceramide, a polyhydroxy acid, gamma-linolenic acid, a fruit acid, sugar cane extract, a skin peel agent, or a cosmeceutically-acceptable salt thereof.

12. The cosmetic method according to any of the preceding claims, wherein said composition is formulated as one or more of: a day and/or night and/or hydrating care composition for the face or the body; an anti-wrinkle or anti-ageing composition; a make-up removing composition; a body milk, a sun protective, artificial sun tanning (self-tanning) or after-sun care composition; and/or a face, skin, body or lip makeup product;

13. The cosmetic method according to any of the preceding claims, wherein said composition is formulated in any suitable manner for application to an individual's skin.

14. The cosmetic method according to any of the preceding claims, wherein said composition is formulated as a lotion, cream, face mask, powder, cosmetic patch, ointment, paste, water-based liquid, oil-based liquid or sprayable liquid.

15. The cosmetic method according to any of the preceding claims, wherein said composition is formulated as a cream or lotion.

16. The cosmetic method according to claim 15, wherein said method for improvement and/or maintenance of skin tone comprises prevention or reduction of skin ageing and/or wrinkling.

17. The method according to any of claims 15-16 wherein said contacting is conducted at least once daily.

18. The method according to any of the claims 15-17 wherein said method further comprises contacting the skin with one or more hormones, plant and/or herbal extracts, moisturizers or humectants, emollients, fragrances, sun-screen actives, anti-wrinkle and/or anti-ageing actives, whitening and/or bleaching actives, sunless tanning actives, preservative and/or antimicrobial actives, anti-acne actives, anti-psoriasis actives, anti-eczema actives, anti-inflammatory actives, vitamin actives, proteins, peptides, amino acids, amino acid derivatives, insect repellants, fungicides, anti-viral agents, anti-cancer agents, anti-hemorrhoid compounds, anti-dandruff compounds, hair-growth stimulating compounds, hair-loss stimulating compounds, nucleic acids, chelating agents, pigments, lipids and/or inorganic salts.

19. The method according to any of claims 15-18 wherein said individual is a post-menopausal, female human being.

20. The method according to any of claims 15-18 wherein said individual is a pre-menopausal, female human being.

21. The method according to any of claims 15-18 wherein said individual is a male human being.

22. The method according to any of claims 15-21 which comprises repeatedly performing said contacting over an extended period of time.

23. Use of an ACE inhibitor and/or angiotensin II receptor antagonist for the preparation of a medicament for the treatment of skin ageing or wrinkling.

24. The use according to claim 23, wherein said skin ageing or wrinkling is considered premature as compared to normal skin ageing and wrinkling.

25. The use according to claim 24 wherein said skin ageing or wrinkling is caused by, or associated with, diabetes mellitus.

26. The use according to claims 24 wherein said skin ageing or wrinkling is caused by, or associated with, smoking or smoke exposure on skin

27. The use according to claim 24, wherein said skin ageing or wrinkling is caused by skin photo-ageing processes.

28. The use according to claim 24, wherein said skin ageing or wrinkling is caused by, or associated with, radiation therapy.

29. The use according to claim 24, wherein said skin ageing or wrinkling is caused by, or associated with, syndrome X.

30. The use according to claim 24, wherein said skin ageing or wrinkling is caused by, or associated with, Hutchinson-Gilford progeria.

31. The use according to claim 24, wherein said skin ageing or wrinkling is caused by, or associated with, Werners syndrome

32. The use according to claim 24, wherein said skin ageing or wrinkling is caused by, or associated with, Kindler syndrome.

33. The use according to claim 24, wherein said skin ageing or wrinkling is caused by, or associated with, corticoid hormone hypersecretion, a vitamin deficiency, administration of glucocorticoids and/or vitamin D and/or derivatives thereof.

34. The use according to any of claims 23-33, wherein the medicament comprises an ACE inhibitor or angiotensin II receptor antagonist or a pharmaceutically acceptable salt thereof.

35. The use according to any claims 23-34 wherein the ACE inhibitor and/or angiotensin II receptor antagonist is according to any of claims 2-6

36. The use according to any of claims 23-35, wherein the medicament is in a formulation for topical administration to the skin

37. The use according to any of claims 23-36 wherein the ACE inhibitor/angiotensin II receptor antagonist is formulated as a lotion, powder, cream, ointment, water-based liquid, paste, oil-based liquid, face mask, skin patch or sprayable liquid.

38. The use according to any of claims 23-37 in combination with one or more of a hormones, a plant and/or herbal extracts, a moisturizers or humectants, an emollients, a fragrances, a sunscreen actives, an anti-wrinkle and/or anti-ageing actives, a whitening and/or bleaching actives, a sunless tanning actives, a preservative and/or antimicrobial actives, an anti-acne actives, an anti-psoriasis actives, an anti-eczema actives, an anti-inflammatory actives, a vitamin active, a protein, a peptide, an amino acid, an amino acid derivative, an insect repellants, a fungicide, an anti-viral agent, an anti-cancer agent, an anti-hemorrhoid compound, an anti-dandruff compound, a hair-growth stimulating compound, a hair-loss stimulating compound, a nucleic acid, a chelating agents, a pigment, a lipid and/or an inorganic salts.

39. The use according to any of claims 23-38, wherein the medicament is administered at least once daily

40. The use according to any of claims 23-39, wherein the medicament is administered at least two times daily.

41. The use according to any of claims 23-40, wherein the medicament is administered in a concentration equivalent of from 0.01 to 100 mg per kg.

42. The use according to any of claims 23-41, wherein the medicament is administered in a concentration equivalent of from 0.1 to 10 mg per kg.

43. Use of an ACE inhibitor and/or angiotensin II receptor antagonist for the preparation of a cosmetic composition for the improvement and/or maintenance of skin tone.

44. The use according to claim 43, wherein said improvement and/or maintenance of skin tone comprises prevention and/or reduction of skin ageing and/or wrinkling.

45. The use according to claims 43-44, wherein said ACE inhibitor and/or angiotensin II receptor antagonist is according to any of claims 2-6

46. The use according to any of claims 43-45, wherein said composition comprises more than one ACE inhibitor or angiotensin II receptor antagonist or a cosmeceutically acceptable salt thereof.

47. The use according to any of claims 43-46 wherein said ACE inhibitor or angiotensin II receptor antagonist is present in an amount according to any of claims 7-8.

48. The use according to any of claims 43-47, wherein said cosmetic composition is formulated in a manner according to any of claims 9-15.
